(19) United States
(12) Patent Application Publication
(10) Pub. No.: US 2021/0022989 A1
(43) Pub. Date: Jan. 28, 2021

(54) METHODS AND COMPOSITIONS FOR PREVENTING IRRITATION OF THE THROAT

(71) Applicant: Practical Elegance, LLC, Cary, NC (US)

(72) Inventors: Lynn Caryl Yanyo, Cary (US); Meredith Payne, Raleigh (US)

(21) Appl. No.: 16/936,017

(22) Filed: Jul. 22, 2020

Related U.S. Application Data
(60) Provisional application No. 62/877,143, filed on Jul. 22, 2019.

(51) Int. Cl. A61K 9/00; A61K 47/44

(52) U.S. Cl. CPC A61K 9/006; A61K 45/06; A61K 47/44

(57) ABSTRACT

Provided herein are protective formulations and compositions suitable for preventing throat irritation in subjects exposed to environmental pollutants. Such compositions can include a carrier oil, optionally a bioactive agent, and a mucilage component. The compositions can be in a topically appliable formulation. Methods of preventing irritation of a throat area of a subject are also provided. Such methods include topically applying a protective formulation or composition as disclosed herein prior to exposure to an environmental condition.

### CROSS-REFERENCE TO RELATED APPLICATIONS

This application claims benefit of U.S. Provisional Patent Application Ser. No. 62/877,143, filed Jul. 22, 2019, herein incorporated by reference in its entirety.

### TECHNICAL FIELD

The presently disclosed subject matter is directed to methods and compositions for preventing irritation of the throat of a subject.

### BACKGROUND

There is presently no product available for the prevention of irritation to the throat. Commonly, people use remedies after the irritation has occurred, using methods such as lozenges, teas, sprays, and tablets of pain reliever. These methods not only take longer to soothe and relieve a sore throat, but can also affect your entire body rather than the localized area.

What is needed is a preventative for irritation of the throat and/or throat area. More particularly, what is needed is compositions and methods for preventing throat irritation prior to exposure to an irritant, such as smoke. Such compositions and methods would be particularly attractive to persons that smoke, vape and the like, in order to prevent irritation of the throat and throat lining (inner throat area) caused by such activities.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this Summary does not list or suggest all possible combinations of such features.

In some embodiments, provided herein are protective formulations for preventing throat irritation in a subject, particularly the inner or interior of the throat, the protective formulation comprising a carrier oil, and a mucilage component, wherein the protective formulation is in a topically appliable formulation. Optionally, in some embodiments such formulations can also comprise a bioactive agent. In some embodiments, the carrier oil is selected from the group consisting of rice bran oil, avocado oil, grapeseed oil, rapeseed oil, olive oil, coconut oil, peanut oil, sunflower seed oil and combinations thereof. In some embodiments, the carrier oil is rice bran oil. In some embodiments, the bioactive agent is selected from the group consisting of Yarrow, Chiretta, Garlic, Aloe Vera, Marshmallow, Arnica, Burdock, Astragalus, Oregon Grape, Borage, Tea, Cayenne, Caraway, Gotu Kola, Cinnamon, German Chamomile, Lemon, Turmeric, Echinacea, Cardamom, Clove, Eucalyptus, Eyebright, Meadowsweet, Licorice, Witch Hazel, Sea Buckthorn, Goldenseal, Elecampane, Lavender, Peppermint, Holy Basil, Evening Primrose, Oregano, Butterbur, Plantain, Rehmannia, Rose, Black Currant, Rosemary, Willow Bark, Sage, Baical Skullcap, Elder, Comfrey, Thyme, Limeflower, Fenugreek, Red Clover, Slippery Elm, Cat's Claw, Heartease, Ginger and combinations thereof. In some embodiments, a viscosity of the carrier oil is from about 0.005 Pa·s to about 0.1 Pa·s. In some embodiments, the mucilage component, or mucilaginous demulcent, is selected from the group consisting of Marshmallow, Hart's-Tongue, Iceland Moss, Kelp, Bladderwrack, Licorice, Flaxseed, Linseed, Loosestrife, Water Lily, Plantain, Psyllium, Solomon's Seal, Peach Tree, Chia, Comfrey, Chickweed, Fenugreek, Slippery Elm, Corn Silk, Okra and combinations thereof.

In some aspects, the bioactive agent comprises about 1% to about 30% by volume of the protective formulation, and the carrier oil comprises about 70% to about 99% by volume of the protective formulation. In some embodiments, the mucilage component comprises about 1% to about 10% by volume of the protective formulation. In some embodiments, the protective formulation is suitable for application to a throat area, particularly the inside or interior of the throat, of a subject. In some embodiments, the protective formulation substantially reduces irritation caused by exposure to smoke or other environmental pollutant. In some embodiments, the protective formulation is configured to remain in place on the throat area of a subject after application for a period of time of at least about 10 minutes, alternatively at least about 15, 20, 25, or 30 minutes or more. In some embodiments, the protective formulation has a substantially neutral taste and/or flavor, and in some embodiments can further comprise a flavoring agent. In some aspects, the protective formulation substantially reduces coughing in a subject inhaling smoke and/or hot vapors.

In other embodiments provided herein are therapeutic compositions for preventing and/or minimizing irritation of the throat of a subject. Such therapeutic compositions can comprise a highly viscous fixed oil, wherein the highly viscous fixed oil comprises an herbal extract, and a mucilaginous demulcent, wherein the therapeutic composition is configured for use as a topically applied therapeutic composition. In some embodiments, the highly viscous fixed oil is selected from the group consisting of rice bran oil, avocado oil, grapeseed oil, rapeseed oil, olive oil, coconut oil, peanut oil, sunflower seed oil and combinations thereof. In some embodiments, the highly viscous fixed oil is rice bran oil. In some embodiments, the herbal extract is selected from the group consisting of Yarrow, Chiretta, Garlic, Aloe Vera, Marshmallow, Arnica, Burdock, Astragalus, Oregon Grape, Borage, Tea, Cayenne, Caraway, Gotu Kola, Cinnamon, German Chamomile, Lemon, Turmeric, Echinacea, Cardamom, Clove, Eucalyptus, Eyebright, Meadowsweet, Licorice, Witch Hazel, Sea Buckthorn, Goldenseal, Elecampane, Lavender, Peppermint, Holy Basil, Evening Primrose, Oregano, Butterbur, Plantain, Rehmannia, Rose, Black Currant, Rosemary, Willow Bark, Sage, Baical Skullcap, Elder, Comfrey, Thyme, Limeflower, Fenugreek, Red Clover, Slippery Elm, Cat's Claw, Heartease, Ginger and combinations thereof. In some embodiments, the mucilaginous demulcent is selected from the group consisting of Marshmallow, Hart's-Tongue, Iceland Moss, Kelp, Bladderwrack, Licorice, Flaxseed, Linseed, Loosestrife, Water Lily, Plantain, Psyllium, Solomon's Seal, Peach Tree, Chia, Comfrey, Chickweed, Fenugreek, Slippery Elm, Corn Silk, Okra and combinations thereof. In some embodiments, the viscosity of the highly viscous fixed oil is about 0.005 Pa·s to about 0.1 Pa·s. In some embodiments, the highly viscous fixed oil comprises about 75% to about 99% by volume of the therapeutic composition, and the mucilaginous demulcent comprises about 1% to about 10% by volume of the composition.

In some embodiments, the therapeutic composition is suitable for application to a throat area of a subject. In some embodiments, the therapeutic composition substantially reduces irritation caused by exposure to smoke or other environmental pollutant. In some embodiments, the therapeutic composition is configured to remain in place on the throat area of a subject after application for a period of time of at least about 10, 15, 20, 25, 30 minutes or more. In some embodiments, the therapeutic composition has a substantially neutral taste and/or flavor. In some embodiments, the therapeutic composition can further comprise a flavoring agent.

Also provided herein are methods of preventing irritation of a throat area of a subject, the methods comprising providing a subject about to be exposed to an environmental condition, applying a protective formulation or therapeutic composition to a throat area of a subject prior to exposure to the environmental condition, wherein the protective formulation or therapeutic composition comprises a carrier oil, and a mucilage component, whereby irritation of the throat of the subject is substantially minimized. In some embodiments, the environmental condition is smoke. In some embodiments, the smoke is from a cigarette, cigar, e-cigarette, bubbler, vaporizer or other inhaled substance. In some embodiments, the protective formulation or therapeutic composition is applied to the subject's throat by slow oral consumption. In some embodiments, the subject is a human subject. Optionally, in some embodiments the protective formulation or therapeutic composition in such methods can also comprise a bioactive agent.

In some embodiments, the protective formulation or therapeutic composition used in the disclosed methods can comprise any of the disclosed protective formulations or therapeutic compositions and various embodiments thereof.

Accordingly, it is an object of the presently disclosed subject matter to provide protective formulations, therapeutic compositions and methods of using the same. These and other objects are achieved in whole or in part by the presently disclosed subject matter. Further, objects of the presently disclosed subject matter having been stated above, other objects and advantages of the presently disclosed subject matter will become apparent to those skilled in the art after a study of the following description, Drawings and Examples.

### DETAILED DESCRIPTION

The presently disclosed subject matter now will be described more fully hereinafter, in which some, but not all embodiments of the presently disclosed subject matter are described. Indeed, the presently disclosed subject matter can be embodied in many different forms and should not be construed as limited to the embodiments set forth herein; rather, these embodiments are provided so that this disclosure will satisfy applicable legal requirements.

### Overview of the Presently Disclosed Subject Matter

In some embodiments, provided herein are protective formulations configured to prevent throat irritation in a subject, the protective formulations comprising a carrier oil, or other suitable carrier medium/substance, and a mucilage component, wherein the protective formulations are configured for use as a topically applied protective agent. In some embodiments, such formulations can further comprise an optional bioactive agent.

In some aspects, provided herein are therapeutic compositions configured to prevent and/or minimize irritation of the throat area, particularly the inner or internal portion of the throat, of a subject, the compositions comprising a highly viscous fixed oil, wherein the highly viscous fixed oil comprises an herbal extract, and a mucilaginous demulcent, wherein the therapeutic compositions are configured for use as a topically applied therapeutic composition. In some aspects, an herbal extract is derived from whole herbs.

The carrier oil and/or highly viscous fixed oil can be selected from rice bran oil, avocado oil, grapeseed oil, rapeseed oil, olive oil, coconut oil, peanut oil, sunflower seed oil and combinations thereof. The bioactive agent and/or herbal extract can be selected from Yarrow, Chiretta, Garlic, Aloe Vera, Arnica, Burdock, Astragalus, Oregon Grape, Borage, Tea, Cayenne, Caraway, Gotu Kola, Cinnamon, German Chamomile, Lemon, Turmeric, Echinacea, Cardamom, Clove, Eucalyptus, Eyebright, Meadowsweet, Witch Hazel, Sea Buckthorn, Goldenseal, Elecampane, Lavender, Peppermint, Holy Basil, Evening Primrose, Oregano, Butterbur, Rehmannia, Rose, Black Currant, Rosemary, Willow Bark, Sage, Baical Skullcap, Elder, Thyme, Limeflower, Red Clover, Cat's Claw, Heartease, Ginger and combinations thereof, with a more comprehensive list provided hereinbelow. As discussed herein, in some aspects the compositions can comprise one or more demulcents (or mucilage, or mucilaginous demulcent), including for example Marshmallow, Licorice, Plantain, Comfrey, Fenugreek, and Slippery Elm, with a more comprehensive list provided hereinbelow.

The protective formulations and/or therapeutic compositions are configured to be applied to the throat or throat area of a subject, and in some aspects are configured to protect the throat of a patient and/or substantially reduce irritation caused by exposure to smoke or other environmental pollutants. Such application can be by oral ingestion or internal consumption by the subject or patient to be treated. Through testing the formula was also found to almost completely eliminate coughing when the formula was applied to the subject's throat before exposure to irritants. The protective formulations and/or therapeutic compositions are configured to remain in place on the throat of a subject after application for a period of time of at least about 10 minutes or more.

Also provided herein are methods of preventing irritation of the throat or inner/internal throat area of a subject. Such methods can comprise providing a subject about to be exposed to an environmental condition, applying a protective formulation or therapeutic composition of any of the above claims prior to exposure to the environmental condition, whereby irritation of the throat of the subject is substantially minimized and coughing is nearly eliminated. In some aspects, the environmental condition is smoke, including for example smoke is from a cigarette, cigar, e-cigarette, vaporizer or other inhaled substance.

### Definitions

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one skilled in the art. While the following terms are believed to be well understood by one of ordinary skill in the art, the following definitions are set forth to facilitate explanation of the presently disclosed subject matter.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value or to an amount of a composition, mass, weight, temperature, time, volume, concentration, percentage, etc., is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

The term “comprising”, which is synonymous with “including” “containing” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C, and D.

As used herein, the terms “treating,” “treatment”, and “to treat” are used to indicate the production of beneficial or desired results, such as to alleviate symptoms, or eliminate the causation of an irritant, a disease or disorder either on a temporary or a permanent basis, slow the appearance of symptoms and/or progression of the disorder, or prevent the condition from occurring. For methods of prevention, a subject to be administered the disclosed compositions is generally a subject at risk for throat irritation and/or dry throat due to exposure to an environmental condition. The terms “treat”, “treatment”, “therapeutic” or “preventative” refer to both therapeutic treatment and prophylactic or preventative measures, wherein the object is to prevent or slow down the development or spread of a condition or symptoms. Beneficial or desired clinical results include, but are not limited to, alleviation of symptoms, diminishment of extent of symptoms, stabilized not worsening) state of disease or condition, delay or slowing of condition progression, amelioration or palliation of the condition state, and remission (whether partial or total).

The term “subject”, “individual”, and “patient” are used interchangeably herein, and refer to an animal, especially a mammal, for example a human, to whom treatment, with a composition as described herein, is provided. The term “mammal” is intended to encompass a singular “mammal” and plural “mammals,” and includes, but is not limited: to humans, primates such as apes, monkeys, orangutans, and chimpanzees; canids such as dogs and wolves; felids such as cats, lions, and tigers; equids such as horses, donkeys, and zebras, food animals such as cows, pigs, and sheep; ungulates such as deer and giraffes; rodents such as mice, rats, hamsters and guinea pigs; and bears.

### DETAILED DESCRIPTION

The presently disclosed subject matter provides methods and compositions for the prevention, mitigation and/or reduction of pain and irritation in the throat of a subject prior to exposure to airborne particles, pollutants and the like, such as for example smoke. The disclosed methods and compositions are configured to protect a subject's throat (or throat area), mouth, nasal cavities and related tissues and linings from irritation and pain that would otherwise result from inhalation of such irritating particles or chemicals or pollutants.

The throat, at least in mammals and other vertebrates, can include the front part of the neck, positioned in front of the vertebra. It contains the pharynx and larynx. The epiglottis is a flap separating the esophagus from the trachea (windpipe) preventing food and drink being inhaled into the lungs. Moreover, the throat contains various blood vessels, pharyngeal muscles, the nasopharyngeal tonsil, the tonsils, the palatine uvula, the trachea, the esophagus, and the vocal cords. Mammalian throats include two bones, the hyoid bone and the clavicle. The throat is sometimes thought to be synonymous for the isthmus of the fauces.

The throat works with the mouth, ears and nose, as well as a number of other parts of the body. The pharynx is connected to the mouth, allowing speech to occur (in humans), and food and liquid to pass down the throat. It is joined to the nose by the nasopharynx at the top of the throat, and to the ear by its Eustachian tube. The throat's trachea carries inhaled air to the bronchi of the lungs. The esophagus carries food through the throat to the stomach. Adenoids and tonsils help prevent infection and are composed of lymph tissue. The larynx contains vocal cords, the epiglottis, and the subglottic larynx. In the larynx, the vocal cords consist of two membranes that act according to the pressure of the air.

The nasal cavity extends from the external opening, the nostrils, to the pharynx (the upper section of the throat), where it joins the remainder of the respiratory system. It is separated down the middle by the nasal septum, a piece of cartilage which shapes and separates the nostrils. Each nostril can be further divided into roof, floor, and walls. The nasal cavity can be divided into the vestibule, respiratory and olfactory sections.

The nasal mucosa, also referred to as a respiratory mucosa, lines the entire nasal cavity, from the nostrils (the external openings of the respiratory system) to the pharynx (the uppermost section of the throat). The external skin of the nose connects to the nasal mucosa in the nasal vestibule. A dynamic layer of mucus overlies the nasal epithelium (the outermost layer of cells of the nasal mucosa). Such tissue is susceptible to irritation by exposure to environmental conditions, elements, and contaminants, e.g. smoke.

The initial one-third of the nasal cavity is lined by stratified squamous epithelium (smooth epithelium consisting of flat surfaced cells), several cell layers thick. The outmost layer of squamous cells overlies a layer of proliferative cells (cell which divide and replicate to form new cells) which is attached to a basement membrane, a network of tough fibers which supports the epithelium. The posterior two-thirds of the cavity is lined with pseudostratified columnar ciliated epithelium (a type of epithelium in which cells arrange themselves in columns and project tiny hairs called cilia) containing goblet cells (mucus producing cells), and which overlies a basement membrane.

The nasal sub-mucosa underlies the basement membrane. This layer is made up of glands which secrete watery substances and mucus, nerves, an extensive network of blood vessels and cellular elements like blood plasma. The entire mucosa is highly concentrated with blood vessels and contains large venous-like spaces; bodies which have a vein-like appearance and swell and congest in response to allergy or infection.

The nasal mucosa plays an important role in mediating immune responses to allergens and infectious particles which enter the nose. The nasal mucosa helps prevent allergens and infections from invading the nasal cavity and spreading to other body structures, for example the lungs. The mucus secreted by and which lines the mucosa provides a physical barrier against invasion by pathogens (harmful microorganisms). The mucus is sticky and traps pathogens when they enter the nasal cavity.

Exposure to pollutants, smoke, dry air and the like can cause irritation of sensitive tissues and linings in the throat, mouth and nasal cavity. For example, when the environment and atmosphere is dry, especially in winter months in some climates, it can create a sensation of dryness and a scratchy feeling in the throat. This is most commonly observed in the morning. Other individuals who also develop throat irritation are those breathing through their mouth because of a congested nose. Environmental pollution is also a common cause of throat irritation. In fact, indoor pollution because of tobacco smoke, medical or recreational marijuana smoke, and the like can be a common cause of throat irritation. Likewise, vaping and the use of e-cigarettes can cause throat irritation. Moreover, smoking tobacco and marijuana can also dry out the supply of mucus/saliva/phlegm in addition to the smoke causing irritation. Other items known to induce throat irritation include alcohol, spicy or hot foods and smokeless tobacco.

Disclosed in some embodiments are compositions comprising a formulation including one or more bioactive agents and one or more carrier oils that have been infused by direct, long-term contact with herbs or mucilage, that are configured to be applied (e.g. by oral consumption) to the anatomical portions and tissues of the throat and related areas in a subject. By way of example and not limitation, the following is a list of plants, being leaf, stem, flower, berry, root, sprout, or juice prepared in essential oil, powder, extract, decoction, tea, infusion, tincture, syrup, capsule, tablet, fixed oil, ointment, cream, or fresh, each of which, or a combination thereof, can be as source of a bioactive agent:

HERBS (Sources of Bioactive Agents):
Yarrow—Achillea Millefolium
Chiretta—Andrographis Paniculata
Garlic—Allium Sativum
Aloe Vera—Aloe Vera
Marshmallow—Althaea officinalis
Arnica—Arnica Montana
Burdock—Arctium lappa
Astragalus—Astragalus membranaceus
Hart's-Tongue—Asplenium Scolopendrium
Oregon Grape—Berberis aquifolium
Borage—Borago officinalis
Tea—Camellia sinensis
Cayenne—Capsicum spp.
Caraway—Carum carv
Gotu Kola—Centella asiatica
Iceland Moss—Cetraria Islandica
Cinnamon—Cinnamomum verum
German Chamomile—Chamomilla recutita
Lemon—Citrus limon
Myrrh—Commiphora Molmol
Turmeric—Curcuma longa
Echinacea—Echinacea angustifolia, purple cone flower
Cardamom—Elettaria cardamomum
Clove—Eugenia caryophyllata
Eucalyptus—Eucalyptus globulus
Eyebright—Euphrasia officinalis
Meadowsweet—Filipendula ulmaria
Galbanum—Ferula Gummosa
Kelp, Bladderwrack—Fucus vesiculosus
Licorice—Glycyrrhiza glabra
Witch Hazel—Hamamelis virginiana
Sea Buckthorn—Hippophae rhamnoides
Goldenseal—Hydrastis canadensis
Elecampane—Inula helenium
Lavender—Lavandula angustifolia
Flaxseed, Linseed—Linum usitatissimum
Loosestrife—Lythrum Salicaria
Peppermint—Mentha x piperita
Arrowroot—Martana Arundinacea
Water Lily—Nymphaea Alba
Holy Basil—Ocimum sanctum, tulsi
Evening Primrose—Oenothera biennis
Oregano—Origanum vulgare, Wild Marjoram
Butterbur—Petasites hybridus
Plantain—Plantago major
Psyllium—Plantago ssp.
Solomon's Seal—Polygonatum Multiflorum
Peach Tree—Prunus Persica
Rehmannia—Rehmannia glutinosa
Rose—Rosa spp.
Black Currant—Ribes nigrum
Rosemary—Rosmarinus officinalis
Willow Bark—Salix alba, white willow
Chia—Salvia hispanica
Sage—Salvia officinalis
Baical Skullcap—Scutellaria baicalensis
Elder—Sambucus nigra
Comfrey—Symphytum officinale
Chickweed—Stellaria media
Tamarind—Tamarindus Indica
Thyme—Thymus vulgaris
Limeflower—Tilia ssp.
Fenugreek—Trigonella foenum-graecum
Red Clover—Trifolium pretense
Slippery Elm—Ulmus rubra
Cat's Claw—Uncaria tomentosa, Una De Gato
Heartease, Johnny Jump Up—Viola tricolor,
Corn Silk—Zea Mays
Ginger—Zingiber officinal

By way of example and not limitation, the following is a list of demulcents. In some embodiments, the disclosed compositions can comprise one or more demulcents. A demulcent is derived from the Latin word for “caress”. A demulcent is an agent that forms a soothing film over a mucous membrane, relieving minor pain and inflammation of the membrane. Demulcents are a classification of plants, sometimes referred to as mucoprotective agents or mucilage-producing agents. In some embodiments a demulcent can also be referred to as a mucilage component, a mucilage and/or a mucilaginouis demulcent, all used interchangeably in some embodiments. Some of the members of this group are food sources, while others come in the form of herbal preparations, tea, dry powder, capsules or tinctures. Demulcent herbs have the ability to act as a protective barrier on irritated or inflamed tissue.

Demulcents:
Marshmallow—Althaea officinalis
Hart's-Tongue—Asplenium Scolopendrium
Iceland Moss—Cetraria Islandica
Kelp, Bladderwrack—Fucus vesiculosus
Licorice—Glycyrrhiza glabra
Flaxseed, Linseed—Linum usitatissimum
Loosestrife—Lythrum Salicaria
Water Lily—Nymphaea Alba
Plantain—Plantago major
Psyllium—Plantago ssp.
Solomon's Seal—Polygonatum Multiflorum
Peach Tree—Prunus Persica
Chia—Salvia hispanica
Comfrey—Symphytum officinale
Chickweed—Stellaria media
Fenugreek—Trigonella foenum-graecum
Slippery Elm—Ulmus rubra
Corn Silk—Zea Mays
Okra—Abelmoschus esculentus

By way of example and not limitation, the following is a list of oils or carrier oils suitable for use in the disclosed compositions.

Oils:
Rice Bran Oil
Avocado Oil
Grapeseed Oil
Rapeseed Oil
Olive Oil
Coconut Oil
Peanut Oil
Sunflower Seed Oil

There is presently no product available for the prevention, reduced occurrence/severity and/or mitigation of irritation to the throat, prior to the irritation occurring. Commonly, people use remedies after the irritation has occurred, using methods such as lozenges, teas, sprays, and tablets of pain reliever. These methods not only take longer to soothe and relieve a sore throat, but can also affect your entire body rather than the localized area.

The compositions and methods disclosed herein can, in some embodiments, prevent irritation of the throat using preparations as disclosed herein. In some embodiments, the disclosed compositions could technically be considered a fixed oil. However, typically, fixed oils are indicated for topical applications after a wound has occurred. Prior to the instant disclosure such fixed oils have not been considered for use as a protectant. Fixed oils are also not typically recommended, even for topical use, as balms and ointments are expected to work better (less greasy) and more efficiently in topical applications.

In some aspects, compositions and methods disclosed herein can include a mucilaginous demulcent designed to coat the tissues and linings of the throat. But when combined with water it was discovered that some mucilaginous demulcents can substantially clear from the throat, in some aspects almost immediately, and therefore offers minimal preventative protection to smoke and other environmental components. This led to the development of a composition with an oil having a high viscosity, which subsequently lead the inventors to oil, including to for example, but not limited to, rice bran oil. Rice bran oil is the highest viscosity edible oil with a neutral taste and no association with nuts (or nut allergies/intolerances). It was discovered that rice bran oil has some effect in prevention of irritation, but it is also low enough in viscosity that it clears the throat in too short of a time, e.g. before a complete cigarette is smoked. In some aspects, the composition can be effective for up to about 15 minutes, sometimes more.

Adding the mucilage to the high viscosity oil thickens the composition even further, creating a product that remains in the throat for a prolonged period of time. This finding was particularly surprising, particularly given that the literature does not recommend putting mucilaginous demulcent (like marshmallow root) directly into oils for extraction. Despite the prior teachings, it was disclosed and disclosed herein that this combination protected a subject's throat (and made the process of inhaling smoke a much more pleasurable experience) far better than oil alone or marshmallow root in water. Every demulcent tested herein in one of the disclosed formulas has only been recommended previously in a decoction or tea form, not in a fixed oil.

In some aspects, a demulcent can be defined as a substance that relieves irritation of the mucous membranes in the mouth by forming a protective film.

In some embodiments, compositions disclosed herein comprise a fixed oil, which in some aspects is defined as a nonvolatile oil, including for example one found in fatty animal tissue and the seeds of various plants, including rice, cotton, corn, flax, sunflowers, peanuts, coconuts, and olives. A fixed oil can also include a natural animal or vegetable oil that is not volatile, and includes a mixture of esters of fatty acids, usually triglycerides.

Mucilage can be defined as a viscous secretion, in some embodiments comprising a polysaccharide substance extracted as a viscous or gelatinous solution from plant roots, seeds, etc.

In some embodiments, the viscosity of the compounds can comprise a measure of its resistance to gradual deformation by shear stress or tensile stress. Example viscosities of some oils and components of the disclosed compounds can be found in Table 1.

Thus, in some embodiments, provided herein are protective formulations configured to prevent throat irritation in a subject, the protective formulations comprising a carrier oil, and a mucilage component, wherein the protective formulations are configured for use as a topically applied protective agent. In some embodiments, such formulations can further comprise an optional bioactive agent.

In some aspects, provided herein are therapeutic compositions configured to prevent and/or minimize irritation of the throat of a subject, the compositions comprising a highly viscous fixed oil, wherein the highly viscous fixed oil comprises an herbal extract, and a mucilaginous demulcent, wherein the therapeutic compositions are configured for use as a topically applied therapeutic composition.

The carrier oil and/or highly viscous fixed oil can be selected from rice bran oil, avocado oil, grapeseed oil, rapeseed oil, olive oil, coconut oil, peanut oil, sunflower seed oil and combinations thereof. The bioactive agent and/or herbal extract can be selected from Yarrow, Chiretta, Garlic, Aloe Vera, Marshmallow, Arnica, Burdock, Astragalus, Oregon Grape, Borage, Tea, Cayenne, Caraway, Gotu Kola, Cinnamon, German Chamomile, Lemon, Turmeric, Echinacea, Cardamom, Clove, Eucalyptus, Eyebright, Meadowsweet, Kelp, Licorice, Witch Hazel, Sea Buckthorn, Goldenseal, Elecampane, Lavender, Flaxseed, Peppermint, Holy Basil, Evening Primrose, Oregano, Butterbur, Plantain, Rehmannia, Rose, Black Currant, Rosemary, Willow Bark, Chia, Sage, Baical Skullcap, Elder, Comfrey, Chickweed, Thyme, Limeflower, Fenugreek, Red Clover, Slippery Elm, Cat's Claw, Heartease, Corn Silk, Ginger and combinations thereof.

The protective formulations can have a viscosity, particularly of the carrier oil, of about 0.005 Pa·s to about 0.1 Pa·s, or in some embodiments about 0.01 Pas to about 0.06 Pa·s. See, for example, Table 1 below.

Table 1 provides mean and standard error for absolute viscosity of the different vegetable oils at different temperatures for a shear rate range of 64.5 to 4835 s⁻¹ and the coefficients of determination (r²) of linear regression for the shear stress and shear rate data.

TABLE 1

| | Temperature | Absolute viscosity | |
|---|---|---|---|
| Oil | (° C.) | (Pa · s) | r² |
| Avocado | 26 | 0.0576 ± 0.0002 | 0.9996-1.0000 |
| | 38 | 0.0364 ± 0.0003 | 0.9996-1.0000 |
| | 50 | 0.0287 ± 0.0006 | 0.9997-1.0000 |
| Canola | 30 | 0.0462 ± 0.0005 | 0.9997-1.0000 |
| | 50 | 0.0245 ± 0.0002 | 0.9993-0.9998 |
| | 90 | 0.0108 ± 0.0004 | 0.9952-0.9982 |
| Grape seed | 26 | 0.0466 ± 0.0003 | 0.9997-1.0000 |
| | 38 | 0.0311 ± 0.0001 | 0.9999-1.0000 |
| | 50 | 0.0227 ± 0.0000 | 0.9995-1.0000 |
| Macadamia nut | 26 | 0.0583 ± 0.0003 | 0.9998-0.9998 |
| | 38 | 0.0394 ± 0.0002 | 0.9996-1.0000 |
| | 50 | 0.0272 ± 0.0012 | 0.9997-0.9998 |
| Olive | 26 | 0.0562 ± 0.0003 | 0.9997-1.0000 |
| | 38 | 0.0341 ± 0.0005 | 0.9994-1.0000 |
| | 50 | 0.0261 ± 0.0003 | 0.9997-1.0000 |
| | 70 | 0.0157 ± 0.0001 | 0.9990-0.9992 |
| Peanut | 26 | 0.0574 ± 0.0007 | 0.9992-0.9996 |
| | 38 | 0.0380 ± 0.0002 | 0.9999-1.0000 |
| | 54 | 0.0236 ± 0.0003 | 0.9998-0.9999 |
| Rapeseed | 26 | 0.0592 ± 0.0011 | 0.9998-0.9999 |
| | 38 | 0.0376 ± 0.0001 | 1.0000-1.0000 |
| | 50 | 0.0305 ± 0.0001 | 0.9972-0.9998 |
| Rice bran | 26 | 0.0593 ± 0.0006 | 0.9996-0.9997 |
| | 38 | 0.0398 ± 0.0001 | 0.9999-1.0000 |
| | 50 | 0.0280 ± 0.0000 | 0.9997-0.9999 |
| Safflower | 26 | 0.0445 ± 0.0003 | 0.9998-1.0000 |
| | 38 | 0.0299 ± 0.0000 | 0.9989-1.0000 |
| | 50 | 0.0239 ± 0.0005 | 0.9992-0.9996 |
| Sesame | 26 | 0.0525 ± 0.0007 | 0.9999-0.9999 |
| | 38 | 0.0351 ± 0.0002 | 1.0000-1.0000 |
| | 50 | 0.0251 ± 0.0002 | 0.9995-0.9999 |
| Soybean | 30 | 0.0405 ± 0.0003 | 0.9993-1.0000 |
| | 50 | 0.0232 ± 0.0001 | 0.9996-0.9998 |
| | 90 | 0.0098 ± 0.0003 | 0.9905-0.9980 |
| Sunflower | 26 | 0.0488 ± 0.0002 | 0.9998-0.9999 |
| | 38 | 0.0323 ± 0.0005 | 0.9998-0.9999 |
| | 50 | 0.0234 ± 0.0001 | 0.9975-0.9993 |
| Walnut | 26 | 0.0429 ± 0.0003 | 0.9998-0.9999 |
| | 38 | 0.0296 ± 0.0002 | 0.9989-1.0000 |
| | 50 | 0.0212 ± 0.0001 | 0.9975-0.9972 |

In the protective formulations the bioactive agent can comprise about 1% to about 30% by volume of the protective formulation, or in some embodiments about 1%, 2%, 3%, 4%, 5%, 10%, 15%, 20%, 25% or 30%. The carrier oil can comprise about 70% to about 99% by volume of the formulation, or in some embodiments about 70%, 75%, 80%, 85%, 90%, 95% or 99%. In some aspects, the mucilage component can comprise about 1% to about 10% by volume of the protective formulation, or in some embodiments about 1%, 2%, 3%, 4%, 5%, 6%, 7%, 8%, 9% or 10%.

The protective formulations and/or therapeutic compositions are configured to be applied to the throat of a subject, and in some aspects are configured to protect the throat of a patient and/or substantially reduce irritation caused by exposure to smoke or other environmental pollutants. The protective formulations and/or therapeutic compositions are configured to remain in place on the throat of a subject after application for a period of time of at least about 10 minutes or more, e.g. about 10 minutes, 15 minutes, 20 minutes, 30 minutes, 1 hour, 2 hours, or more.

Also provided herein are methods of preventing irritation of the throat of a subject. Such methods can comprise providing a subject about to be exposed to an environmental condition, applying a protective formulation or therapeutic composition of any of the above claims prior to exposure to the environmental condition, whereby irritation of the throat of the subject is substantially minimized. In some aspects, the environmental condition is smoke, including for example smoke from a cigarette, cigar, e-cigarette, vaporizer or other inhaled substance.

In some aspects, the protective formulation or therapeutic composition can be applied to the subject's throat by any suitable or appropriate method, including for example by oral consumption. In some aspects, before smoking or exposure to irritants, a user can pour a single dose (e.g. about 10 ml) of the disclosed composition into the mouth slowly and swallow slowly to allow for maximum effect. Once taken, it may in some embodiments be recommended to avoid food or drink until the exposure to irritants is finished. In some aspects, and by way of example only, the composition can be packaged into single-dose packets that are easily and conveniently transportable and usable.

### Examples

The following examples are included to further illustrate various embodiments of the presently disclosed subject matter. However, those of ordinary skill in the art should, in light of the present disclosure, appreciate that many changes can be made in the specific embodiments which are disclosed and still obtain a like or similar result without departing from the spirit and scope of the presently disclosed subject matter.

The present disclosure features methods and compositions for the prevention of pain and irritations in the throat prior to exposure to airborne particles, pollutants and the like. The disclosed methods and compositions are configured to protect a subject's throat, mouth, nasal cavities and related tissues and linings from irritation and pain that would otherwise result from inhalation of such irritating particles or chemicals or pollutants. Experiments were conducted to confirm the effectiveness of these methods and compositions, the results of which are summarized in Tables 2 and 3 below.

TABLE 2
Bubbler (average of 20 subjects)

| | Throat Feeling Scale | | | Coughing Scale | |
|---|---|---|---|---|---|
| | | Pretreatment | | | Pretreatment |
| | | Demulcent | Pretreatment | | Demulcent |
| | Control | Oil | Oil | Control | Oil |
| All | 7.6 | 8.9 | 8.8 | 8 | 0.5 |
| Male | 7.5 | 9 | 9 | 7.8 | 0.8 |
| Female | 7.7 | 8.8 | 8.5 | 8.3 | 0 |

TABLE 3
Cigarette (average of 47 subjects)

| | Throat Feeling Scale | | | Coughing Scale | |
|---|---|---|---|---|---|
| | | Pretreatment | | | Pretreatment |
| | | Demulcent | Pretreatment | | Demulcent |
| | Control | Oil | Oil | Control | Oil |
| All | 6.9 | 8.7 | 8.0 | 10.8 | 0.7 |
| Male | 7.3 | 8.8 | 8.6 | 8.9 | 0.5 |
| Female | 6.6 | 8.6 | 7.8 | 12.0 | 0.8 |

Sixty-seven individual subjects participated in the testing (20 with bubbler (Table 2) and 47 with cigarette (Table 3)). Prior to starting each experiment, each subject was to confirm their throats felt normal with no prior pain or irritation. Each subject was asked to take three consistent inhales of marijuana before treatment (as a control) and then three more consistent inhales of marijuana with a pretreatment (either demulcent in oil or just oil alone). Every subject smoked the same strain of marijuana out of the same glass piece or a rolled cigarette. The glass piece used was a bubbler, meaning it was a glass piece that has a bowl that flows into a water chamber for filtration and then travels through the rest of the piece to the subject's mouth. The bowl is packed with marijuana flower, and the subject lights the marijuana on fire, and the smoke travels through the water and into the subject's mouth, throat and then lungs. The rolled cigarette was lit by the subject and the first inhalation was the lighting of the cigarette. Each subject determined how long the inhale was, as long as all 6 inhales were consistent. After each inhale, the subject was asked to rate the feeling of his/her throat on the following scale (Table 4):

TABLE 4
Throat Feeling Scale

| Scale | Feeling/sensation |
|---|---|
| 10 | No Pain/ Normal |
| 9 | |
| 8 | Slight Soreness/Slight Pain |
| 7 | |
| 6 | Noticeable Pain |
| 5 | |
| 4 | Very Uncomfortable |
| 3 | |
| 2 | Intense/Throbbing Pain |
| 1 | Gagging |

The subjects did not know which pre-treatment they were taking. The two pre-treatments that were tested were rice bran oil alone and rice bran oil that had been infused with marshmallow root (demulcent). The marshmallow root was soaked in the rice bran oil for 3 weeks and then the marshmallow root solids were filtered out, so the two pre-treatments looked identical to the naked eye. Each subject took 10 ml of pre-treatment in a shot glass and drank it before the latter sequence of 3 inhales. Each subject was asked to refrain from drinking or eating anything during each sequence of 3 inhales, but was permitted to drink water between sequences.

The observer counted and recorded the number of times the subject coughed during the experiment after each inhale. This led to a surprising discovery; most of subjects coughed during the initial control sequence of inhales. However, once those subjects took the pre-treatment demulcent oil, coughing was virtually non-existent. Even subjects who did not cough during the control sequence said they felt an improvement on their urge to cough.

These results confirm that in human subjects oil and a combination of demulcent and oil are better than having no protection at all. For more irritating delivery of smoke (cigarette vs bubbler), the demulcent oil provides improved protection over oil alone. These results also confirm that in human subjects oil and a combination of oil and demulcent used before exposure to smoke can significantly reduce coughing.

### REFERENCES

All references listed herein including but not limited to all patents, patent applications and publications thereof, scientific journal articles, and database entries are incorporated herein by reference in their entireties to the extent that they supplement, explain, provide a background for, or teach methodology, techniques, and/or compositions employed herein.
Marieb E N. Essential of Human Anatomy and Physiology. (7th edition). San Francisco: Benjamin Cummings; 2003.
Mann M D. Gustatory and Olfactory senses. Chapter 10. The nervous system in action. [online]. 2008. University of Nebraska Medical Centre.

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A protective formulation for preventing throat irritation in a subject, the protective formulation comprising:
a carrier oil; and
a mucilage component,
wherein the protective formulation is in a topically or internally applicable formulation.

2. The protective formulation of claim 1, further comprising a bioactive agent.

3. The protective formulation of claim 1, wherein the carrier oil is selected from the group consisting of rice bran oil, avocado oil, grapeseed oil, rapeseed oil, olive oil, coconut oil, peanut oil, sunflower seed oil and combinations thereof.

4. The protective formulation of claim 3, wherein the carrier oil is rice bran oil.

5. The protective formulation of claim 2, wherein the bioactive agent is selected from the group consisting of Yarrow, Chiretta, Garlic, Aloe Vera, Marshmallow, Arnica, Burdock, Astragalus, Oregon Grape, Borage, Tea, Cayenne, Caraway, Gotu Kola, Cinnamon, German Chamomile, Lemon, Turmeric, Echinacea, Cardamom, Clove, Eucalyptus, Eyebright, Meadowsweet, Licorice, Witch Hazel, Sea Buckthorn, Goldenseal, Elecampane, Lavender, Peppermint, Holy Basil, Evening Primrose, Oregano, Butterbur, Plantain, Rehmannia, Rose, Black Currant, Rosemary, Willow Bark, Sage, Baical Skullcap, Elder, Comfrey, Thyme, Limeflower, Fenugreek, Red Clover, Slippery Elm, Cat's Claw, Heartease, Ginger and combinations thereof.

6. The protective formulation of claim 1, wherein a viscosity of the carrier oil is from about 0.005 Pa·s to about 0.1 Pa·s.

7. The protective formulation of claim 2, wherein the bioactive agent comprises about 1% to about 25% by volume of the protective formulation, and the carrier oil comprises about 75% to about 99% by volume of the protective formulation.

8. The protective formulation of claim 1, wherein the mucilage component comprises about 1% to about 10% by volume of the protective formulation.

9. The protective formulation of claim 1, wherein the protective formulation is suitable for application to an inner throat area of a subject, wherein the protective formulation substantially reduces irritation caused by exposure to smoke or other environmental pollutant.

10. The protective formulation of claim 9, wherein the protective formulation is configured to remain in place on the inner throat area of a subject after application for a period of time of at least about 10, 15, 20, 25, 30 minutes or more.

11. The protective formulation of claim 1, wherein the protective formulation has a substantially neutral taste and/or flavor, wherein the protective formulation further comprises a flavoring agent.

12. A therapeutic composition for preventing and/or minimizing irritation of the throat of a subject, the composition comprising:
a highly viscous fixed oil, wherein the highly viscous fixed oil comprises an herbal extract; and
a mucilaginous demulcent,
wherein the therapeutic composition is configured for use as a topically or internally applied therapeutic composition.

13. The therapeutic composition of claim 12, wherein the highly viscous fixed oil is selected from the group consisting of rice bran oil, avocado oil, grapeseed oil, rapeseed oil, olive oil, coconut oil, peanut oil, sunflower seed oil and combinations thereof.

14. The therapeutic composition of claim 12, wherein the highly viscous fixed oil is rice bran oil.

15. The therapeutic composition of claim 12, wherein the herbal extract is selected from the group consisting of Yarrow, Chiretta, Garlic, Aloe Vera, Arnica, Burdock, Astragalus, Oregon Grape, Borage, Tea, Cayenne, Caraway, Gotu Kola, Cinnamon, German Chamomile, Lemon, Turmeric, Echinacea, Cardamom, Clove, Eucalyptus, Eyebright, Meadowsweet, Witch Hazel, Sea Buckthorn, Goldenseal, Elecampane, Lavender, Peppermint, Holy Basil, Evening Primrose, Oregano, Butterbur, Rehmannia, Rose, Black Currant, Rosemary, Willow Bark, Sage, Baical Skullcap, Elder, Thyme, Limeflower, Red Clover, Cat's Claw, Heartease, Ginger and combinations thereof.

16. The therapeutic composition of claim 12, wherein the mucilaginous demulcent is selected from the group consisting of Marshmallow, Hart's-Tongue, Iceland Moss, Kelp, Bladderwrack, Licorice, Flaxseed, Linseed, Loosestrife, Water Lily, Plantain, Psyllium, Solomon's Seal, Peach Tree, Chia, Comfrey, Chickweed, Fenugreek, Slippery Elm, Corn Silk, Okra and combinations thereof.

17. The therapeutic composition of claim 12, wherein the viscosity of the highly viscous fixed oil is about 0.005 Pa·s to about 0.1 Pa·s.

18. The therapeutic composition of claim 12, wherein the highly viscous fixed oil comprises about 75% to about 99% by volume of the therapeutic composition, and the mucilaginous demulcent comprises about 1 to about 10% by volume of the composition.

19. The therapeutic composition of claim 12, wherein the therapeutic composition is suitable for application to a throat area of a subject, wherein the therapeutic composition substantially reduces irritation caused by exposure to smoke or other environmental pollutant.

20. The therapeutic composition of claim 19, wherein the therapeutic composition is configured to remain in place on the inner throat area of a subject after application for a period of time of at least about 10, 15, 20, 25, 30 minutes or more.

21. The therapeutic composition of claim 12, wherein the therapeutic composition has a substantially neutral taste and/or flavor, wherein the therapeutic composition further comprises a flavoring agent.

22. A method of preventing irritation of an inner throat area of a subject, the method comprising:
providing a subject about to be exposed to an environmental condition;
applying a protective formulation or therapeutic composition to a throat area of a subject prior to exposure to the environmental condition, wherein the protective formulation or therapeutic composition comprises:
a carrier oil; and
a mucilage component,
whereby irritation of the inner throat area of the subject is substantially minimized.

23. The method of claim 22, wherein the environmental condition is smoke.

24. The method of claim 23, wherein the smoke is from a cigarette, cigar, e-cigarette, bubbler, vaporizer or other inhaled substance.

25. The method of claim 22, wherein the protective formulation or therapeutic composition is applied to the subject's inner throat area by slow oral consumption.

26. The method of claim 22, wherein the formulation or therapeutic composition further comprises a bioactive agent.
